(19) United States
(12) Patent Application Publication
(10) Pub. No.: US 2025/0072720 A1
(43) Pub. Date: Mar. 6, 2025

(54) EXPANSION UNIT OF ENDOSCOPE AND ADAPTER DEVICE OF ENDOSCOPE

(71) Applicant: FUJIFILM Corporation, Tokyo (JP)

(72) Inventor: Yoshihiro UEDA, Ashigarakami-gun (JP)

(73) Assignee: FUJIFILM Corporation, Tokyo (JP)

(21) Appl. No.: 18/825,907

(22) Filed: Sep. 5, 2024

Related U.S. Application Data
(60) Provisional application No. 63/536,710, filed on Sep. 6, 2023.

(51) Int. Cl. A61B 1/00

(52) U.S. Cl. CPC A61B 1/00087; A61B 1/00121

(57) ABSTRACT

An expansion unit of an endoscope includes: a distal end member that is configured to be able to be held at a distal end part of the endoscope; a first tubular member that extends from the distal end member; a cylindrical member that has a first opening, that is connected to a base end part of the first tubular member opposite to a side of the distal end member, and a second opening; and a connecting member that connects the second opening and a treatment tool inlet port of the endoscope.

### CROSS REFERENCE TO RELATED APPLICATION

This application is based on and claims priority from U.S. Provisional Application No. 63/536,710 filed on Sep. 6, 2023, the entire disclosure of which is incorporated herein by reference.

### BACKGROUND OF THE INVENTION

### 1. Field of the Invention

The technology of the present disclosure relates to an expansion unit of an endoscope and an adapter device of the endoscope.

### 2. Description of the Related Art

JP1984-48601U (JP-S59-48601U) discloses an endoscope device consisting of: an endoscope body including a distal end part including an observation window or the like, a hand operating part, and an intra-body cavity insertion part that connects the distal end part and the hand operating part; and a channel forming tube through which a treatment tool such as forceps having a first attachment and detachment unit that is attachably and detachably attached to the distal end part and a second attachment and detachment unit that is attachably and detachably attached to the hand operating part is inserted, the first attachment and detachment unit being provided on a distal end side and the second attachment and detachment unit being provided on a base end side.

JP1997-47426A (JP-H9-47426A) discloses an attachable/detachable channel type endoscope comprising: an endoscope body that has an elongated insertion part in which an observation optical system with an observation window provided at a distal end part is built and an operation part that is connected to a hand side of the insertion part; and a hollow channel that is partially attachably and detachably mounted along the insertion part, that has one end which is open near the distal end part, the other end provided with a connecting portion with a suction source, and a middle part having a branch portion, and that has a treatment tool insertion port at one end branched by the branch portion, in which the channel includes a connecting portion that can be separated near the branch portion.

JP1999-216104A (JP-H11-216104A) discloses a suction device for an endoscope, which is provided in an endoscope and is provided with a valve mounting portion provided in a middle of an endoscope suction path with a distal end that is open to a distal end portion of an insertion part, in which a suction valve is mounted on the valve mounting portion to switch between a suction operation state in which the endoscope suction path is connected to a suction source and a non-operation state in which the suction source communicates with the atmosphere, the suction device for an endoscope comprising: an endoscopic external-suction passage that is extended along the insertion part; and a second suction valve that is attachably and detachably mounted on the valve mounting portion and that is switchable between the non-operation state in which the suction source communicates with the atmosphere and the suction operation state in which the communication with the atmosphere is cut off and the suction source is connected to the endoscopic external-suction passage.

JP2005-131107A discloses an external channel comprising: a flexible first lumen; a flexible second lumen that extends along the first lumen; and a fixing unit that attachably and detachably fixes the first lumen and the second lumen to an insertion part of an endoscope.

JP2010-63483A discloses an auxiliary tool that is used in combination with an endoscope having an insertion part to be inserted into a body cavity from one external nostril and that includes an insertion part which assists a function of the endoscope by being inserted into the body cavity from the other external nostril.

JP2018-68517A discloses a fixing device that fixes an endoscope capable of observing an operative site in a body cavity using an endoscope having an insertion part to be inserted into the body cavity and an endoscope body attached to one end of the insertion part, in which the fixing device includes a holder body attached to a fixing stand placed on a floor and holding the endoscope body in an axially rotatable manner, and the holder body includes an attachment/detachment mechanism that allows the endoscope body to be attached to and detached from the holder body while keeping the insertion part inserted in the body cavity.

### SUMMARY OF THE INVENTION

The present disclosure provides a technology capable of increasing the number of channels of an endoscope in a retrofitted manner.

An expansion unit of an endoscope according to one aspect of the technology of the present disclosure comprises: a distal end member that is configured to be able to be held at a distal end part of the endoscope; a first tubular member that extends from the distal end member; a cylindrical member that has a first opening, that is connected to a base end part of the first tubular member opposite to a side of the distal end member, and a second opening; and a connecting member that connects the second opening and a treatment tool inlet port of the endoscope.

An adapter device of an endoscope according to another aspect of the technology of the present disclosure comprises: a first engagement portion that attachably and detachably engages with an operation part of the endoscope; and a second engagement portion that attachably and detachably engages with an expansion unit that adds a channel to the endoscope.

According to the technology of the present disclosure, it is possible to increase the number of channels of the endoscope.

### BRIEF DESCRIPTION OF THE DRAWINGS

FIG. 1 is a diagram showing a schematic configuration of an endoscope device 100 which is one aspect of a technology of the present disclosure.

FIG. 2 is an exploded perspective view schematically showing a schematic configuration of an expansion unit 1A according to one aspect of the technology of the present disclosure.

FIG. 3 is a schematic view partially showing a state in which the expansion unit 1A shown in FIG. 2 is mounted on an endoscope 1 via an adapter device 60.

FIG. 4 is a schematic cross-sectional view of a cylindrical member 40.

FIG. 5 is a schematic cross-sectional view of a first connecting portion 51.

FIG. 6 is a perspective view of a distal end member 20 and a first tubular member 30 in the expansion unit 1A as viewed in a direction different from a direction in FIG. 2.

FIG. 7 is a schematic cross-sectional view taken along a line A-A in FIG. 2.

FIG. 8 is a view of the distal end member 20 and the first tubular member 30 shown in FIG. 6 as viewed in a direction X1.

FIG. 9 is a perspective view showing a configuration of the adapter device 60.

FIG. 10 is a perspective view of the adapter device 60 as viewed in a direction different from a direction in FIG. 9.

FIG. 11 is a diagram showing an example of a mounting state of the cylindrical member 40 and the adapter device 60 on the endoscope 1.

FIG. 12 is a diagram showing a state in which the cylindrical member 40 is rotated by 180 degrees from the state of FIG. 11.

FIG. 13 is a diagram showing a state in which the adapter device 60 and an operation part 11 are fixed by a fixing tool 80.

FIG. 14 is a side view showing a state in which the cylindrical member 40 is engaged with the adapter device 60 mounted on the operation part 11.

FIG. 15 is a perspective view showing a configuration example of an attachment 70.

FIG. 16 is a perspective view showing a state in which grip forceps with a bending function are additionally used in the endoscope 1.

FIG. 17 is a diagram showing a modification example of the expansion unit 1A.

### DESCRIPTION OF THE PREFERRED EMBODIMENTS

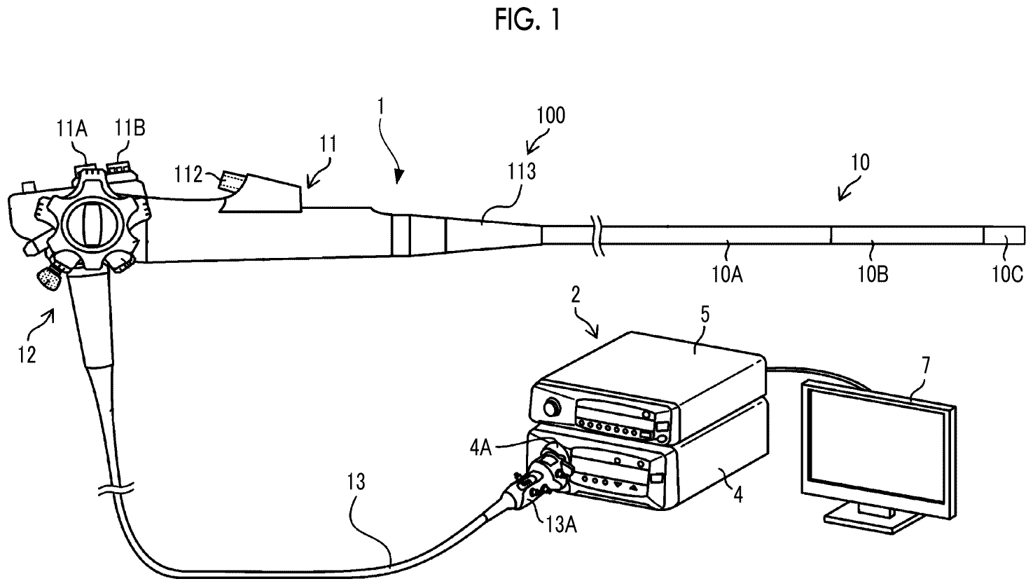

FIG. 1 is a diagram showing a schematic configuration of an endoscope device 100 which is one aspect of a technology of the present disclosure. The endoscope device 100 comprises an endoscope 1, a body part 2 consisting of a light source device 4 and a processor device 5 to which the endoscope 1 is connected, and a display device 7 that displays a captured image obtained by imaging with the endoscope 1, and the like.

The endoscope 1 comprises an insertion part 10 that is an elongated instrument extending in one direction and that is to be inserted into a subject, an operation part 11 that is provided at a base end part of the insertion part 10 and that is provided with an operation member for performing an observation mode switching operation, an imaging and storing operation, a forceps operation, an air/water supply operation, a suction operation, an electric scalpel operation, or the like, an angle knob 12 that is provided adjacent to the operation part 11, and a universal cord 13 including a connector portion 13A that attachably and detachably connects the endoscope 1 to a connecting portion 4A of the light source device 4.

The operation part 11 is provided with a treatment tool inlet port 112 into which a treatment tool for sampling a biological tissue such as a cell or a polyp is introduced. Although not shown in FIG. 1, inside the operation part 11 and the insertion part 10, various channels, such as a treatment tool channel, an air/water supply channel, and a suction channel, through which the treatment tool introduced from the treatment tool inlet port 112 is inserted are provided. The operation part 11 includes a suction button 11A, an air/water supply button 11B, and the like.

The insertion part 10 is configured of a soft part 10A having flexibility, a bendable part 10B provided at a distal end of the soft part 10A, and a distal end part 10C that is provided at a distal end of the bendable part 10B and that is harder than the soft part 10A. An imaging element and an imaging optical system are built in the distal end part 10C.

The bendable part 10B is configured to be bendable by a rotational movement operation of the angle knob 12. The bendable part 10B can be bent in any direction and at any angle depending on a site of a subject or the like for which the endoscope 1 is used, and the distal end part 10C can be directed in a desired direction.

A light guide configured by bundling a plurality of optical fibers is provided inside the endoscope 1, extending from the distal end part 10C of the insertion part 10 to the connector portion 13A. Light generated by the light source device 4 is introduced into the light guide from the connector portion 13A, advances to the distal end part 10C, and is emitted to the subject from an illumination window provided at the distal end part 10C.

The universal cord 13 includes the suction channel. For example, a suction device is provided in or externally attached to the light source device 4, and the suction device and the suction channel are connected to each other. In a case where the suction button 11A included in the operation part 11 is operated, a negative pressure generated by the suction device is connected to a distal end of the endoscope 1, and a fluid or the like is sucked from the distal end of the endoscope 1 into the suction channel and is collected by the suction device.

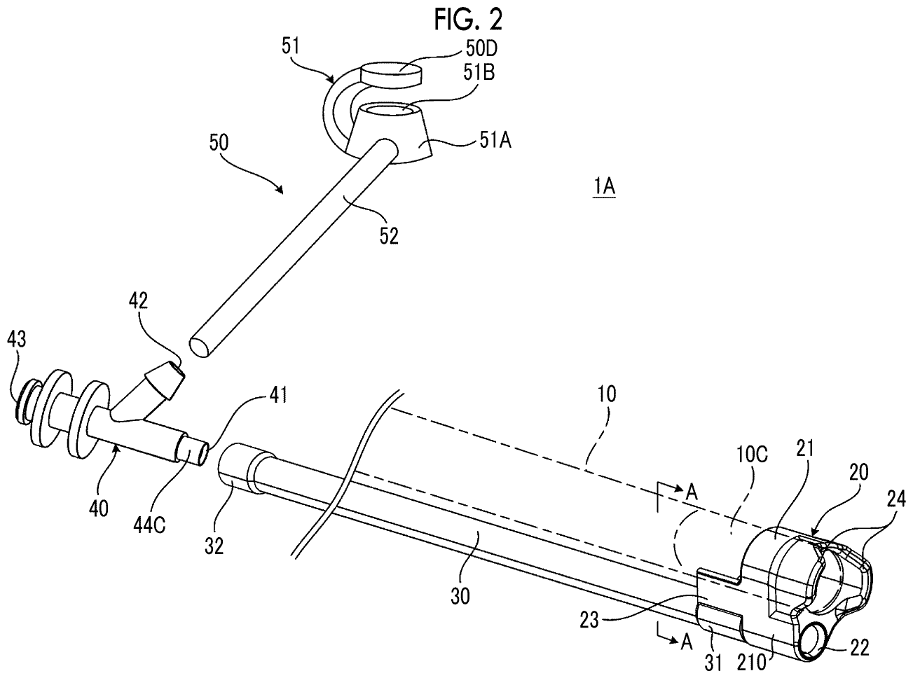

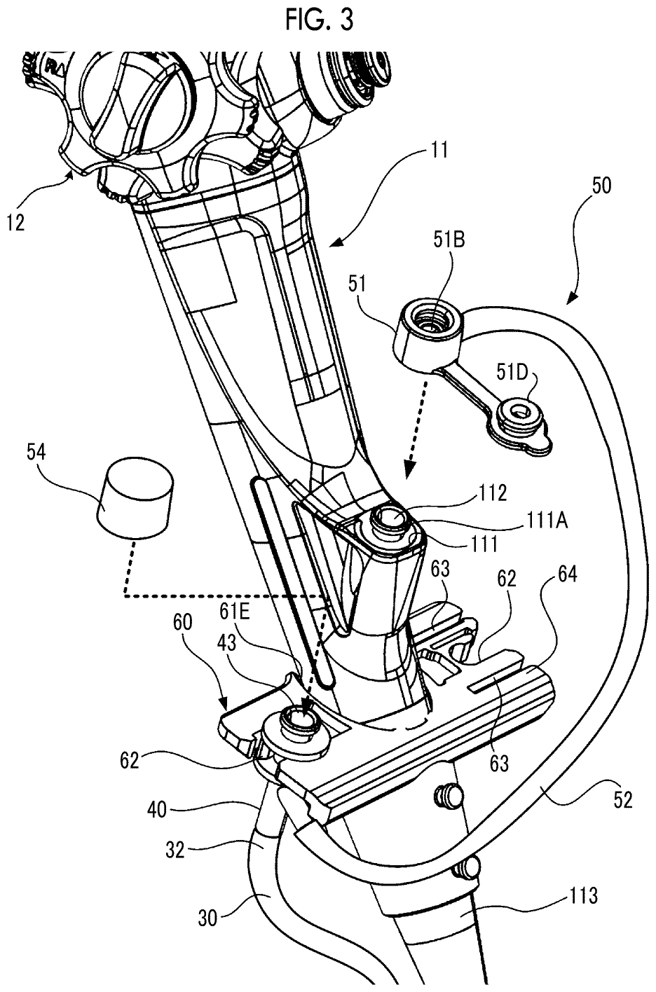

Next, a configuration of an expansion unit that adds a channel to the endoscope 1 shown in FIG. 1 and an adapter device for mounting the expansion unit on the endoscope 1 will be described. FIG. 2 is an exploded perspective view schematically showing a schematic configuration of an expansion unit 1A according to one aspect of the technology of the present disclosure. FIG. 3 is a schematic view partially showing a state in which the expansion unit 1A shown in FIG. 2 is mounted on the endoscope 1 via an adapter device 60.

As an example, the expansion unit 1A adds the treatment tool channel and the suction channel with respect to the endoscope 1. As shown in FIG. 2, the expansion unit 1A comprises a distal end member 20 that is configured to be able to be held at the distal end part 10C of the endoscope 1, a first tubular member 30 that extends from the distal end member 20, a cylindrical member 40 connected to a base end part 32 of the first tubular member 30 opposite to the distal end member 20 side, and a connecting member 50 that connects the cylindrical member 40 and the treatment tool inlet port 112 of the endoscope 1.

As shown in FIG. 3, the adapter device 60 is used by being engaged with an end part 113 of the operation part 11 on a distal end side of the endoscope 1. The expansion unit 1A is mounted on the endoscope 1 via the adapter device 60 by engaging the cylindrical member 40 with the adapter device 60. As shown in FIG. 3, the treatment tool inlet port 112 of the endoscope 1 is configured by a mouthpiece 111A that is fitted into an opening 111 provided in a housing of the operation part 11.

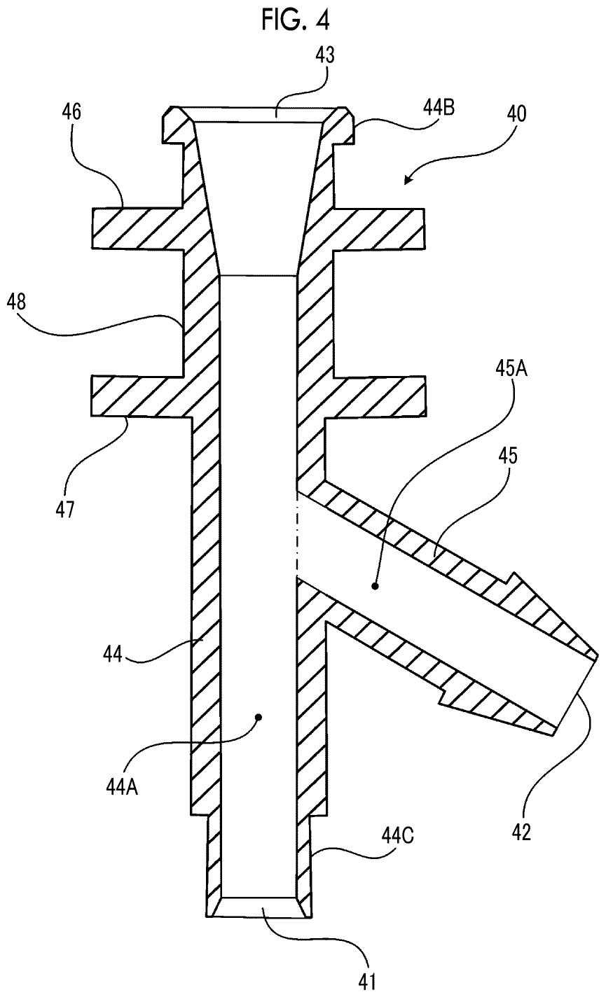

FIG. 4 is a schematic cross-sectional view of the cylindrical member 40. The cylindrical member 40 has a cylindrical main portion 44 that forms a linear passage 44A and a cylindrical branch portion 45 that forms a branch path 45A branched from the passage 44A and extending in a direction intersecting a direction in which the passage 44A extends. In the present specification, the term “passage” refers to a space through which at least one of the treatment tool or the fluid can pass.

The main portion 44 has a first opening 41 on one end side in an axial direction and has a third opening 43 on the other end side in the axial direction. A columnar space from the first opening 41 to the third opening 43 forms the passage 44A. The branch portion 45 protrudes obliquely from an outer peripheral surface of the main portion 44 with respect to an axis of the main portion 44, and has a second opening 42 at an end part on a side opposite to the main portion 44 side. The second opening 42 communicates with the passage 44A, and a columnar space leading from the second opening 42 to the passage 44A forms the branch path 45A.

The cylindrical member 40 further has a first flange portion 46 and a second flange portion 47 arranged in the axial direction of the main portion 44 on the outer peripheral surface of the main portion 44. The first flange portion 46 and the second flange portion 47 are provided in a portion between the third opening 43 and the branch portion 45 in the axial direction of the main portion 44. A portion of the main portion 44 between the first flange portion 46 and the second flange portion 47 and the first flange portion 46 and the second flange portion 47 constitute an engaged portion 48 that is engageable with the adapter device 60.

A shape of an end part 44B (an upper part with respect to the first flange portion 46 in FIG. 4), at which the third opening 43 is formed, of the main portion 44 of the cylindrical member 40 is preferably the same as a shape of the mouthpiece 111A provided in the operation part 11. An end part 44C, at which the first opening 41 is formed, of the main portion 44 is configured to have a smaller diameter than the other portions of the main portion 44. As shown in FIG. 3, the base end part 32 of the first tubular member 30 is connected to the end part 44C having a small diameter. The end part 44C is fixed to the base end part 32 by welding or adhesion in a state of being inserted into the base end part 32. The first tubular member 30 has flexibility so as to be deformable to follow the insertion part 10 of the endoscope 1.

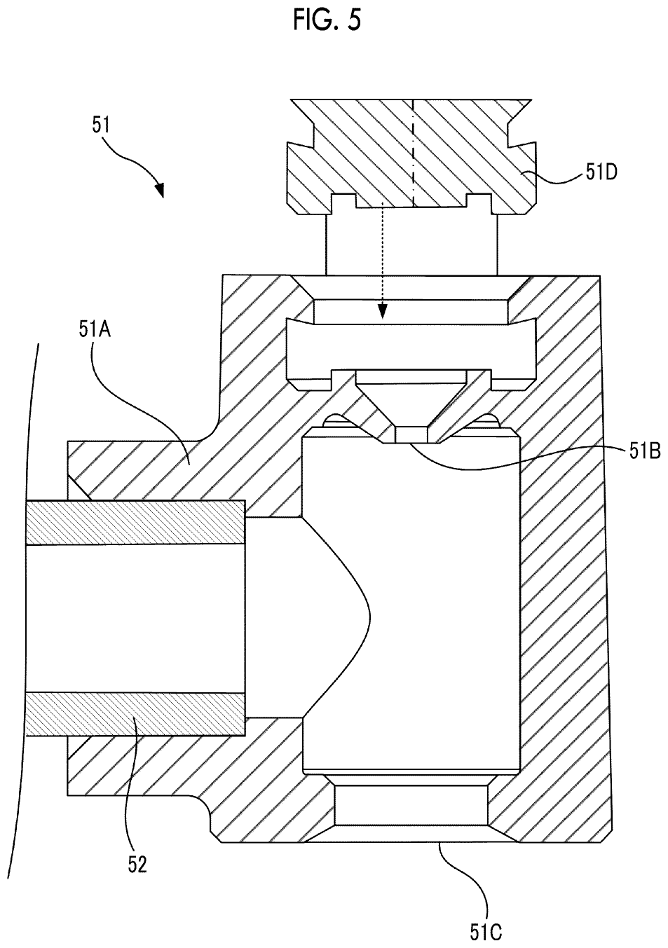

As shown in FIG. 2, the connecting member 50 has a first connecting portion 51 that is connected to the treatment tool inlet port 112 (in other words, the mouthpiece 111A) of the endoscope 1, and a second tubular member 52 having one end connected to the second opening 42 of the cylindrical member 40 and the other end connected to the first connecting portion 51. The second tubular member 52 preferably has flexibility. The second tubular member 52 and the cylindrical member 40 may be fixed to each other by welding, adhesion, or the like, or may be attachable to and detachable from each other by fitting, press-fitting, or the like. FIG. 5 is a schematic cross-sectional view of the first connecting portion 51. As shown in FIG. 5, the first connecting portion 51 comprises a sleeve part 51A connected to the treatment tool inlet port 112 (mouthpiece 111A), and a lid part 51D that covers an opening 51B among openings 51B and 51C arranged in the sleeve part 51A in the axial direction. An inner space of the sleeve part 51A is connected to an inside of the second tubular member 52. The opening 51C has a shape that engages with the mouthpiece 111A of the endoscope 1.

In a state in which the opening 51C of the first connecting portion 51 is engaged with the mouthpiece 111A and the opening 51B is opened by the lid part 51D, the treatment tool can be introduced into the treatment tool channel inside the endoscope 1 via the opening 51B and the treatment tool inlet port 112. In a state in which the opening 51C of the first connecting portion 51 is engaged with the mouthpiece 111A and the opening 51B is closed by the lid part 51D, a negative pressure is generated inside the sleeve part 51A by operating the suction button 11A, and a fluid inside the second tubular member 52 can be collected by the above-described suction device. That is, the expansion unit 1A can function as an additional suction channel.

The lid part 51D is not essential in the first connecting portion 51. For example, as disclosed in JP2005-198834A, the sleeve part 51A and the lid part 51D are integrally formed, and a slit (see a one-dot chain line in FIG. 5) that can communicate with the opening 51B is provided in the lid part 51D. An inside of the sleeve part 51A is always kept in a sealed state, and a structure can be adopted in which the slit is expanded to allow the treatment tool to be inserted into the inside of the sleeve part 51A in a case where the treatment tool is inserted. The sleeve part 51A need only be configured to be capable of generating a negative pressure inside in a case where the suction device is operated (in a case where the suction button 11A is operated).

In a case where the shape of the end part 44B of the main portion 44 of the cylindrical member 40 is the same as the shape of the mouthpiece 111A, the first connecting portion 51 can also be connected to the end part 44B. In a case where the first connecting portion 51 is connected to the end part 44B, the third opening 43 and the second opening 42 of the cylindrical member 40 can be connected to each other by the connecting member 50. In a state in which the opening 51C of the first connecting portion 51 is engaged with the end part 44B of the cylindrical member 40 and the opening 51B is opened by the lid part 51D, the treatment tool can be introduced into the first tubular member 30 via the opening 51B, the third opening 43, and the first opening 41. That is, the first tubular member 30 in the expansion unit 1A can function as an additional treatment tool channel.

The first connecting portion 51 may have a structure in which the lid part 51D and the sleeve part 51A are integrated. Even in a case where this structure is adopted, the treatment tool can be inserted into the treatment tool inlet port 112 while ensuring airtightness of the treatment tool inlet port 112 to maintain the suction function, as long as a slit or a pinhole is provided on a top surface of the integrated connecting portion 51.

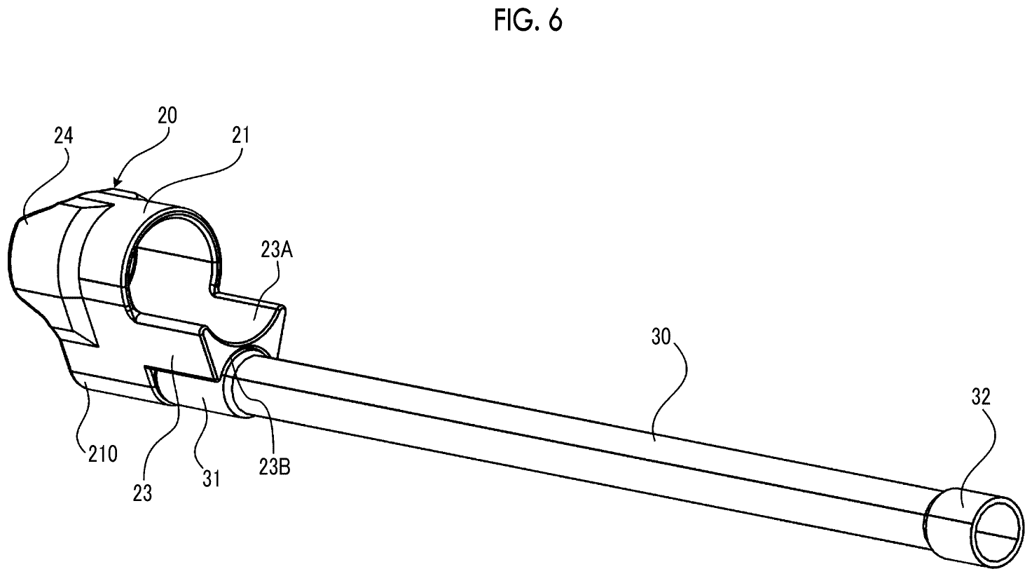

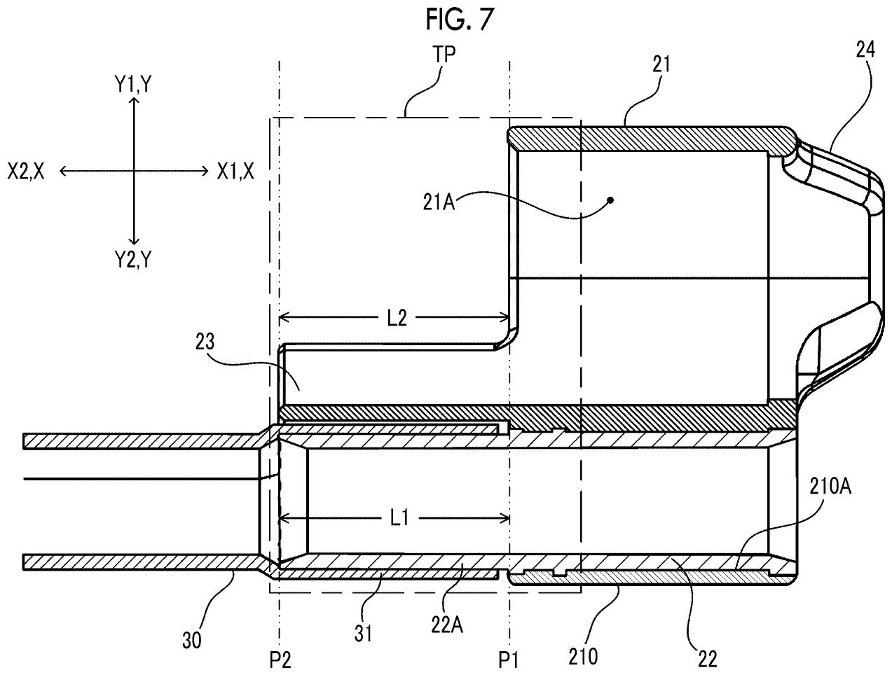

FIG. 6 is a perspective view of the distal end member 20 and the first tubular member 30 in the expansion unit 1A as viewed in a direction different from a direction in FIG. 2. FIG. 7 is a schematic cross-sectional view taken along a line A-A in FIG. 2.

The distal end member 20 has an engagement portion 21 that engages with the distal end part 10C of the endoscope 1, and a pipe line 22 that is radially disposed adjacent to the distal end part 10C in a state in which the engagement portion 21 is engaged with the distal end part 10C and that communicates with the first tubular member 30.

As shown in FIG. 7, the engagement portion 21 has a cylindrical shape that forms a cylindrical space 21A. An inner diameter of the engagement portion 21 is configured to be substantially the same as or slightly smaller than an outer diameter of the distal end part 10C, and the distal end member 20 is engaged with the distal end part 10C by inserting the distal end side of the distal end part 10C into the space 21A. FIG. 7 shows the axial direction of the endoscope 1 in a state in which the distal end member 20 is engaged with the distal end part 10C as a direction X, and shows a radial direction of the endoscope 1 as a direction Y. Of the directions X, a direction from the base end to the distal end of the endoscope 1 is shown as a direction X1, and a direction opposite to the direction X1 is shown as a direction X2. The direction X1 is an imaging direction of the subject by the imaging element built in the distal end part 10C. In FIG. 7, one of the directions Y is shown as a direction Y1, a direction opposite to the direction Y1 is shown as a direction Y2, and a position P1 of an edge of the engagement portion 21 on the direction X2 side is shown.

The distal end member 20 has a bulging part 210 that bulges from the engagement portion 21 in the direction Y2. As shown in FIG. 7, a hole portion 210A penetrating in the direction X is formed in the bulging part 210. The pipe line 22 is provided in the hole portion 210A. The pipe line 22 is formed of, for example, a cylindrical metal pipe. The pipe line 22 has a pipe line extending part 22A (a portion on a left side of the position P1 in FIG. 7) that extends further in the direction X2 than the engagement portion 21. A distal end part 31 of the first tubular member 30 is connected to the pipe line extending part 22A. The distal end part 31 of the first tubular member 30 is fixed to an outer peripheral surface of the pipe line extending part 22A by adhesion, welding, or the like. FIG. 7 shows a position P2 of an edge of the pipe line extending part 22A on the direction X2 side.

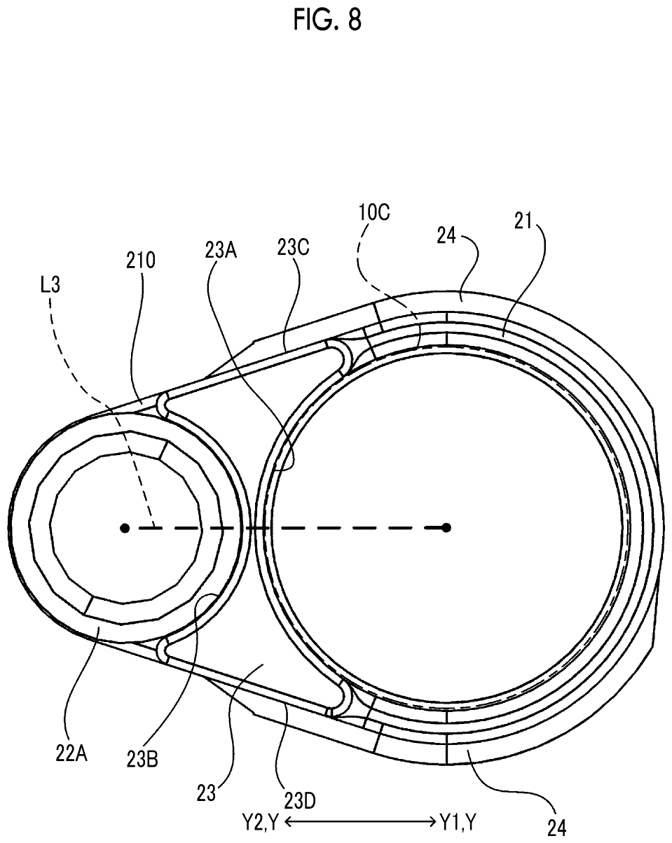

The distal end member 20 has a distal end member extending part 23 that extends from the engagement portion 21 in the direction X2. FIG. 8 is a view of the distal end member 20 and the first tubular member 30 shown in FIG. 6 as viewed in the direction X1. As shown in FIG. 8, the distal end member extending part 23 is provided between the distal end part 10C that is engaged with the engagement portion 21 and the pipe line extending part 22A, and is provided adjacent to the distal end part 10C and the pipe line extending part 22A. In addition, the distal end member extending part 23 is provided across a line segment L3 connecting a center of the distal end part 10C that is engaged with the engagement portion 21 and a center of the pipe line extending part 22A in a case of being viewed in the direction X. A side surface of the distal end member extending part 23 includes a first curved surface 23B that faces the outer peripheral surface of the pipe line extending part 22A and extends along the outer peripheral surface, a second curved surface 23A that faces an outer peripheral surface of the distal end part 10C that is engaged with the engagement portion 21 and extends along the outer peripheral surface, a plane 23C that connects one edge of the first curved surface 23B and one edge of the second curved surface 23A, and a plane 23D that connects the other edge of the first curved surface 23B and the other edge of the second curved surface 23A.

As shown in FIG. 7, the distal end member 20 has a protruding portion 24 that protrudes from the engagement portion 21 in the imaging direction (the direction X1 in FIG. 7) of the endoscope 1 that is engaged with the engagement portion 21. As shown in FIG. 2, two protruding portions 24 are provided at positions facing each other with the space 21A interposed therebetween. It is preferable that the two protruding portions 24 are provided to face each other with an extension line of the line segment L3 in FIG. 8 interposed therebetween. The protruding portion 24 is provided, so that even in a case where a distal end of the distal end member 20 comes into contact with an organ of the subject, it is possible to prevent the organ and a distal end surface of the distal end part 10C that is engaged with the engagement portion 21 from coming into contact with each other. In addition, it is preferable that the protruding portions 24 have a symmetrical shape with respect to the extension line of the line segment L3. As a result, the protruding portion has the same shape regardless of whether the pipe line 22 is disposed on a left or right side of an endoscope screen, and the endoscopy or operation can be performed in the same environment regardless of the disposition of the pipe line 22. In general, since the treatment tool channel of the endoscope is disposed on a lower side of the endoscope screen, there is an advantage in that a vertical direction position of the treatment tool on the endoscope screen can be aligned by disposing the attachment position of the pipe line 22 on either the left or right side of the endoscope screen. In addition, in the endoscope having a function of supplying water to a front of the endoscope, it is preferable that water can be supplied to a position close to an axis of the treatment tool channel of the endoscope, and the pipe line 22 can be disposed on either the left or right side, taking into consideration a position of an outlet of the channel that supplies water to the front. In addition, it is desirable that a height of the protruding portion 24 is relatively low on the line segment L3. As a result, it is difficult for water to accumulate between the protruding portion 24 and the distal end part 10C, and even in a case where the water accumulates, the water can be quickly removed by sucking from the pipe line 22, and a good endoscopic view can be maintained. In FIG. 7, a relatively low portion in the protruding portion 24 is at the same height as an end surface of the pipe line 22, but as long as the relatively low portion is lower than the most protruding portion, it may be higher or lower than the height of the end surface.

The engagement portion 21, the distal end member extending part 23, and the protruding portion 24 are formed of, for example, a molded product such as a resin, a rubber, and an elastomer. The pipe line 22 can be integrally formed with a molded product by insert molding, but the pipe line 22 may be separately formed. In a case where the inner diameter of the engagement portion 21 is smaller than the outer diameter of the distal end part 10C, it is preferable that the material of the molded product is flexible like rubber so that the distal end part 10C and the engagement portion 21 can be fixed by pushing the distal end part 10C and widening the inner diameter of the engagement portion 21. On the other hand, in order for the protruding portion 24 to prevent the distal end part 10C from coming into contact with the organ, a certain degree of rigidity is required. In order to satisfy both of these requirements, a hardness is preferably about Shore A 30 to 70. In addition, a height of the most protruding portion in the protruding portion 24 is preferably about 2 to 5 mm from an end surface of the distal end part 10C, and a thickness in the radial direction is preferably about 1 to 3 mm. With the configuration according to the above range, especially in use of a treatment tool with a bending function, there is an advantage in that, in a case where the treatment tool rotates at a position very close to the end surface of the distal end part 10C, the protruding portion 24 is deformed appropriately even though the protruding portion 24 and the treatment tool interfere with each other, and the operation of the treatment tool is less likely to be hindered.

Outer peripheries of the distal end member 20 and the distal end part 10C are wound with a medical pressure-sensitive adhesive tape TP (see FIG. 7) such as a surgical tape, so that the distal end member 20 is more securely held by the distal end part 10C. Specifically, the pressure-sensitive adhesive tape TP is wound such that the pipe line extending part 22A and an outer portion of the engagement portion 21 of the distal end part 10C are integrated, so that a position of the distal end member 20 is more firmly held with respect to the distal end part 10C.

In a case where the distal end member extending part 23 exists, as shown in FIG. 8, a gap between the distal end part 10C and the pipe line extending part 22A can be filled with the distal end member extending part 23. In a case where the reinforcement is performed with the pressure-sensitive adhesive tape TP, the gap is filled with the distal end member extending part 23, so that the distal end part 10C and the pipe line extending part 22A can be firmly fixed to each other. In addition, since the plane 23C and the plane 23D of the distal end member extending part 23 can come into contact with the pressure-sensitive adhesive tape TP in a plane, peeling of the pressure-sensitive adhesive tape TP can be prevented.

In addition, since the distal end member extending part 23 is provided on both sides of the line segment L3, the distal end part 10C and the pipe line extending part 22A can be firmly fixed to each other as compared to a configuration in which the distal end member extending part 23 is provided only on one side of the line segment L3. In addition, since the distal end member extending part 23 is provided on both sides of the line segment L3, it is possible to prevent the pipe line extending part 22A and the distal end part 10C from coming into contact with each other, and to improve durability of the distal end member 20 and the endoscope 1. From the viewpoint of durability, it is preferable that a length L2 (see FIG. 7) of the distal end member extending part 23 in the direction X is equal to or greater than a length L1 (see FIG. 7) of the pipe line extending part 22A in the direction X. In this way, it is possible to effectively prevent the pipe line extending part 22A and the distal end part 10C from coming into contact with each other as compared to a configuration in which the length L2 is less than the length L1.

In addition, since the distal end member extending part 23 has the second curved surface 23A, the second curved surface 23A serves as a guide in a case where the distal end part 10C is engaged with the engagement portion 21, and the engagement between the distal end part 10C and the distal end member 20 can be easily performed. In addition, substantially the entire surface of the second curved surface 23A comes into contact with the outer peripheral surface of the distal end part 10C, thereby obtaining stable fixation. In addition, since the distal end member extending part 23 has the first curved surface 23B, it is possible to facilitate the work of connecting the distal end part 31 of the first tubular member 30 to the pipe line extending part 22A.

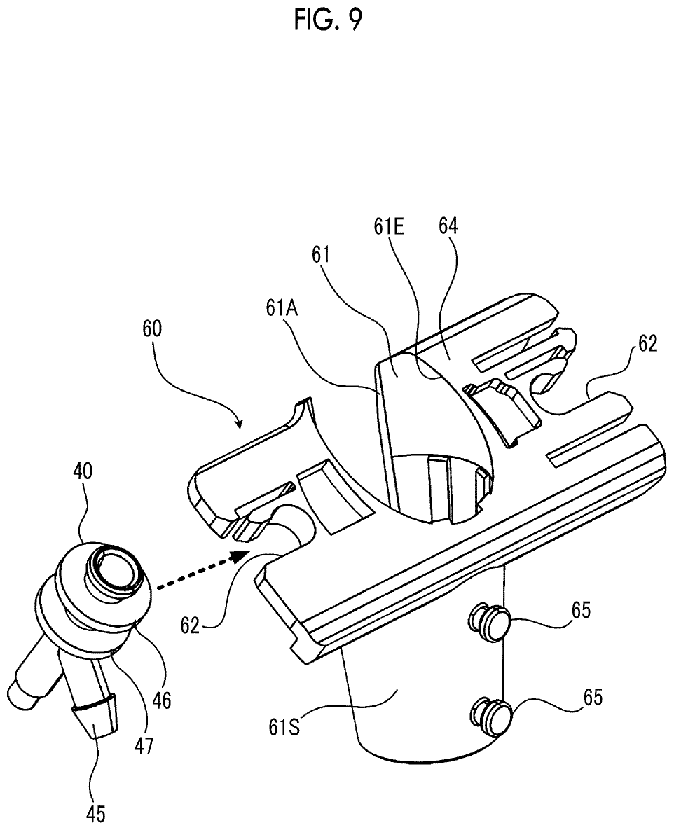

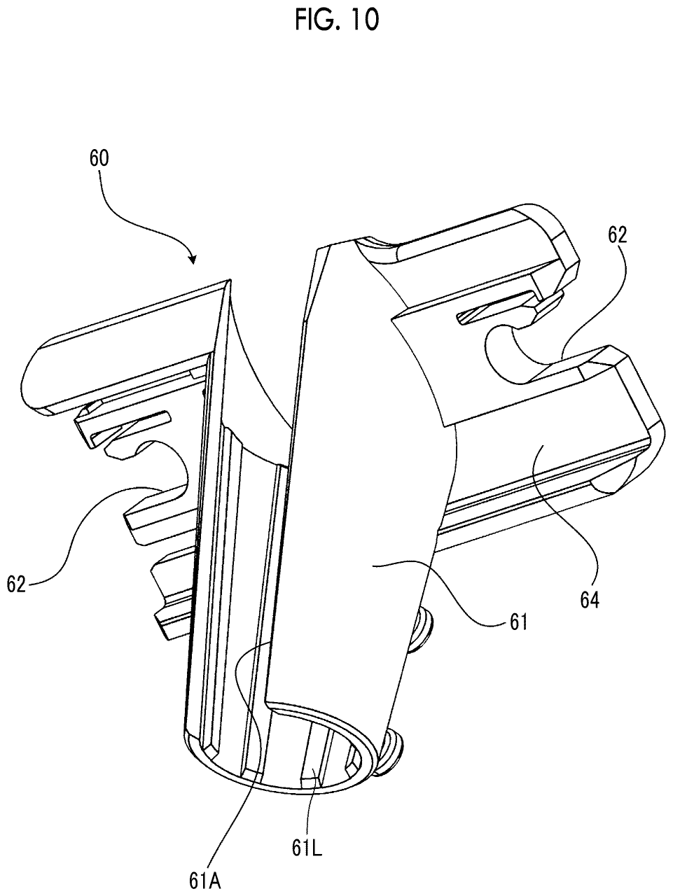

FIG. 9 is a perspective view showing a configuration of the adapter device 60. FIG. 10 is a perspective view of the adapter device 60 as viewed in a direction different from a direction in FIG. 9. The adapter device 60 comprises a first engagement portion 61 that attachably and detachably engages with the operation part 11 of the endoscope 1, and a second engagement portion 62 that attachably and detachably engages with the cylindrical member 40 of the expansion unit 1A. The first engagement portion 61 is formed of a cylindrical body, and has a substantially C-shaped cross section in which an opening 61A extending from one end to the other end in the axial direction is provided on an outer peripheral surface of the cylindrical body. The first engagement portion 61 can be quickly engaged with the operation part 11, for example, by passing the insertion part of the endoscope from a side surface direction through the above-described substantially C-shaped notch portion and then sliding the insertion part in the axial direction with respect to the operation part 11. The first engagement portion 61 may be formed of a cylindrical body in which the opening 61A is closed. In this case, by passing the insertion part 10 of the endoscope 1 to an inside of the cylindrical body from the distal end side, the end part 113 and the first engagement portion 61 can be engaged with each other. The first engagement portion 61 may be formed of either a cylindrical body having an opening on the outer peripheral surface or a cylindrical body having no opening on the outer peripheral surface.

The adapter device 60 has a flange portion 64 that protrudes from the first engagement portion 61. The flange portion 64 is provided to protrude from an outer edge of one opening 61E of two openings in the axial direction of the first engagement portion 61. As shown in FIG. 3, the opening 61E is located on the base end side of the endoscope 1 in a state in which the adapter device 60 is attached to the operation part 11, among the two openings in the axial direction of the first engagement portion 61.

Two second engagement portions 62 are provided on the flange portion 64, and the two second engagement portions 62 are disposed to face each other with the first engagement portion 61 interposed therebetween. In the example of FIG. 9, the second engagement portion 62 includes a hole portion that penetrates the flange portion 64 in a thickness direction and an elastically deformable claw portion provided in the hole portion, and is configured to be able to engage with the engaged portion 48 of the cylindrical member 40 through a snap fit. In a state in which the cylindrical member 40 is engaged with the second engagement portion 62, the flange portion 64 is interposed between the first flange portion 46 and the second flange portion 47, so that the cylindrical member 40 and the adapter device 60 are firmly engaged with each other. In FIG. 3, the cylindrical member 40 is engaged with the second engagement portion 62 on a left side in the figure, but the cylindrical member 40 can also be engaged with the second engagement portion 62 on a right side in the figure.

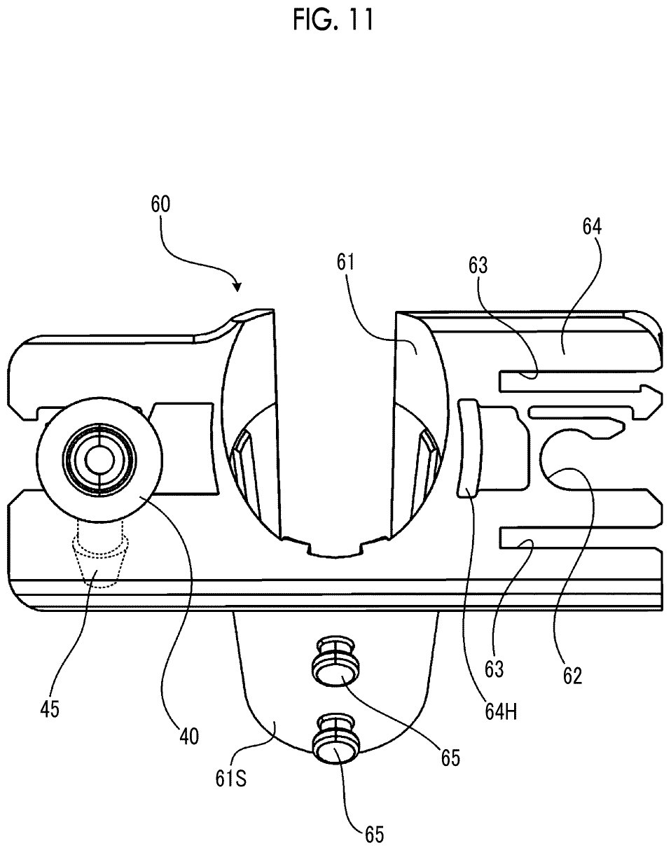

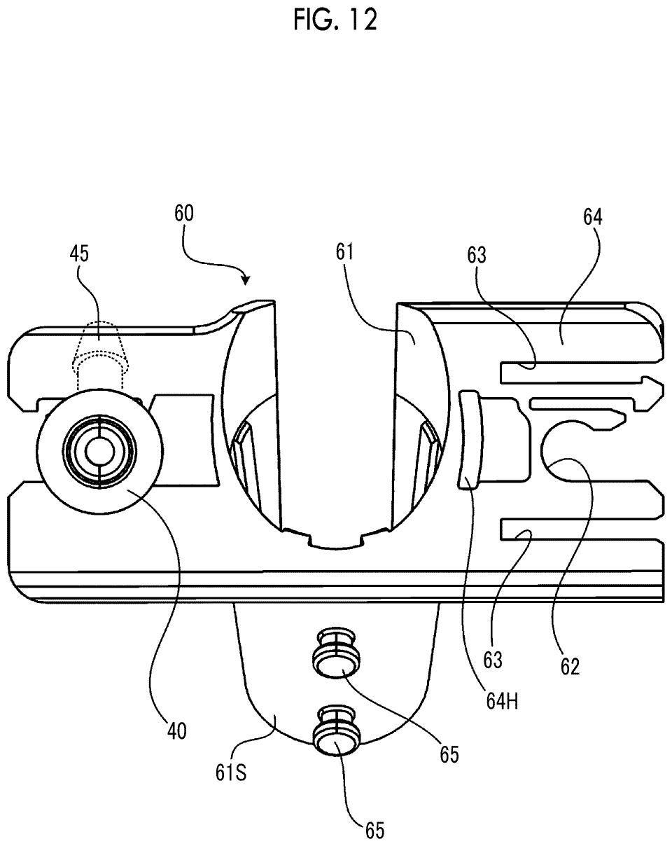

The cylindrical member 40 is rotatable about an axis of the main portion 44 in a state of being engaged with the second engagement portion 62. Therefore, an orientation of the branch portion 45 with respect to the adapter device 60 can be freely changed. For example, as shown in FIG. 11, a state shown in FIG. 12 can be obtained by rotating the branch portion 45 by 180 degrees from a state in which the cylindrical member 40 is engaged with the second engagement portion 62, so that interference between the branch portion 45 or the second tubular member 52 and the operation part 11 can be prevented.

As shown in FIGS. 11 and 12, the flange portion 64 is provided with an oval flange hole portion 64H that penetrates the flange portion 64 in the thickness direction between one (the right second engagement portion 62 in the figure) of the two second engagement portions 62 and the first engagement portion 61. In addition, two protrusions 65 are provided on an outer peripheral surface 61S of the first engagement portion 61 on a side opposite to the opening 61A. Since the adapter device 60 is configured to be attached to and detached from the endoscope 1, it is preferable that both the adapter device 60 and the endoscope 1 can be firmly fixed to each other so that relative positions of the adapter device 60 and the endoscope 1 do not shift. As an example of a tool for performing such fixation, a fixing tool 80 of a band-shaped body made of an elastic body such as rubber is used. In addition, for example, by providing a plurality of ribs 61L (see FIG. 10), which extend in the axial direction, on an inner peripheral surface of the first engagement portion 61, fixation in a rotation direction can be made more reliable.

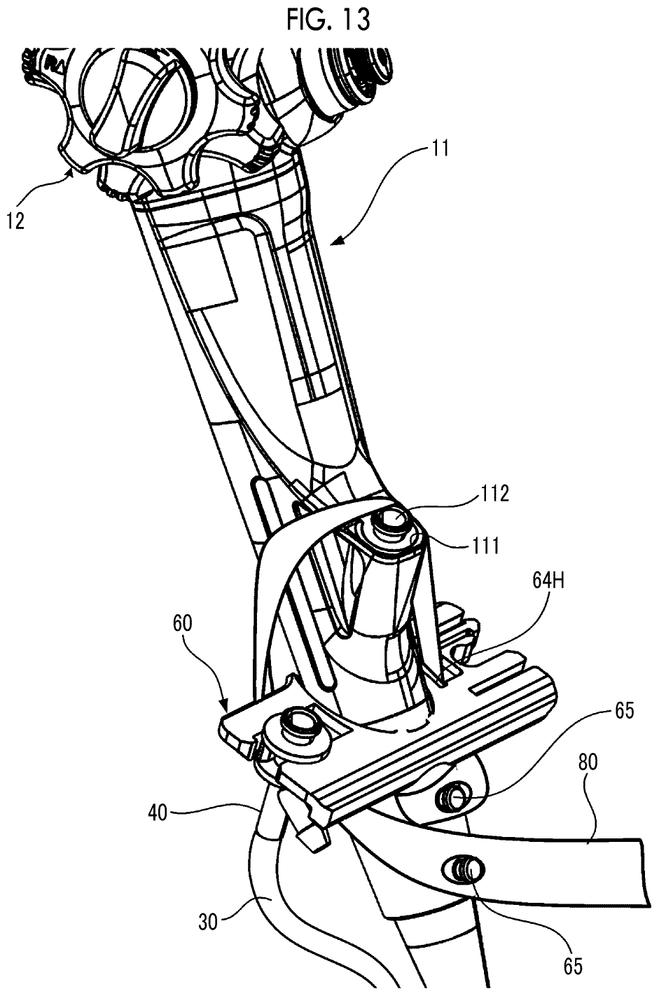

FIG. 13 is a diagram showing a state in which the adapter device 60 and the operation part 11 are fixed by the fixing tool 80. The fixing tool 80 is, for example, a rubber band. One end of the fixing tool 80 is locked to the protrusion 65 on a lower side in the figure of the adapter device. The fixing tool 80 extends from a position of the protrusion 65 on the lower side in the figure to a position of the opening 111 through a left side in the figure of the first engagement portion 61 and a lower side in the figure of the flange portion 64, and is hooked onto a part of the operation part 11. The fixing tool 80 hooked onto a part of the operation part 11 is locked to the protrusion 65 on an upper side in the figure through the flange hole portion 64H. As described above, the protrusion 65 constitutes a locking portion to which the fixing tool 80 can be locked. In addition, since the flange hole portion 64H is provided in the flange portion 64, the adapter device 60 and the operation part 11 can be more firmly fixed to each other as compared to a configuration in which the fixing tool 80 is locked only by the protrusion 65.

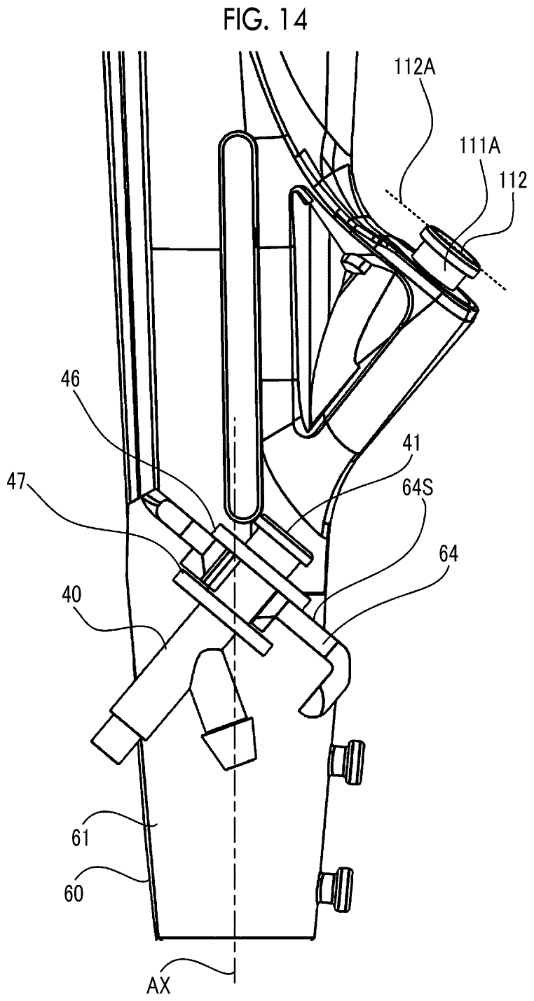

FIG. 14 is a side view showing a state in which the cylindrical member 40 is engaged with the adapter device 60 mounted on the operation part 11. As shown in FIG. 14, the flange portion 64 is provided to be inclined with respect to an axis AX of the first engagement portion 61. In the state of FIG. 14 in the flange portion 64, a surface 64S on the base end side of the endoscope 1 has a shape along an opening surface 112A (shown by a broken line for convenience in FIG. 14) of the mouthpiece 111A. The surface 64S being along the opening surface 112A means that an angle between the surface 64S and the opening surface 112A is equal to or less than a threshold value (for example, 45 degrees), and includes a configuration in which the surface 64S and the opening surface 112A are parallel to each other. Since the surface 64S is along the opening surface 112A, the first opening 41 of the cylindrical member 40 engaged with the flange portion 64 and the treatment tool inlet port 112 are oriented in the same direction. Therefore, in a case where it is assumed that a treatment tool is introduced into each of the first opening 41 and the treatment tool inlet port 112 to perform treatment, the two treatment tools have the same introduction direction, and the treatment can be efficiently performed.

In the state shown in FIG. 14, the flange portion 64 is disposed on the distal end side of the endoscope 1 with respect to the treatment tool inlet port 112. With this configuration, in a case where the treatment tool is introduced into each of the first opening 41 and the treatment tool inlet port 112 to perform the treatment, it is possible to prevent interference between the two treatment tools, and to efficiently perform the treatment.

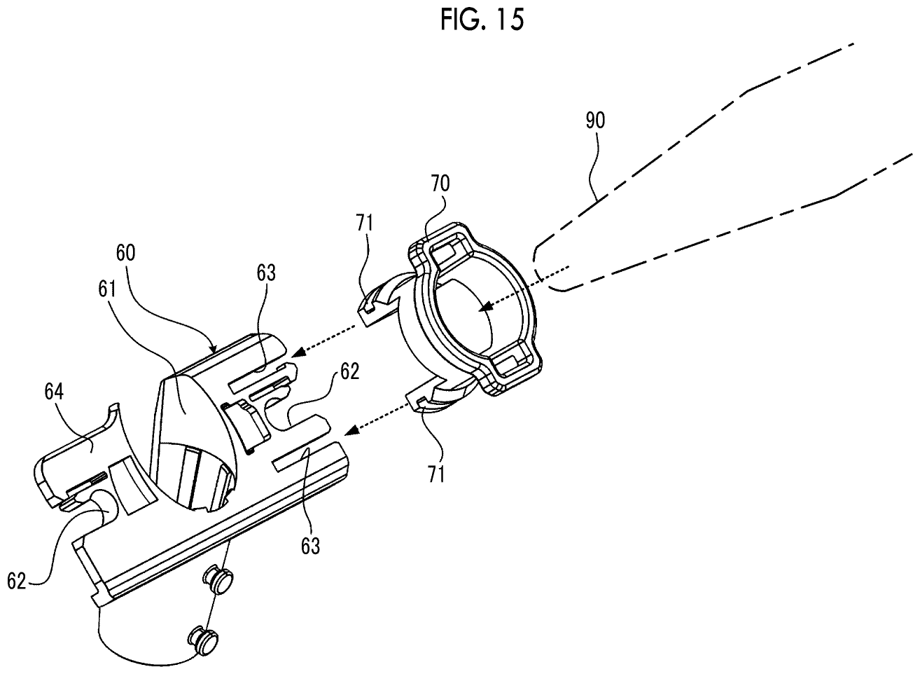

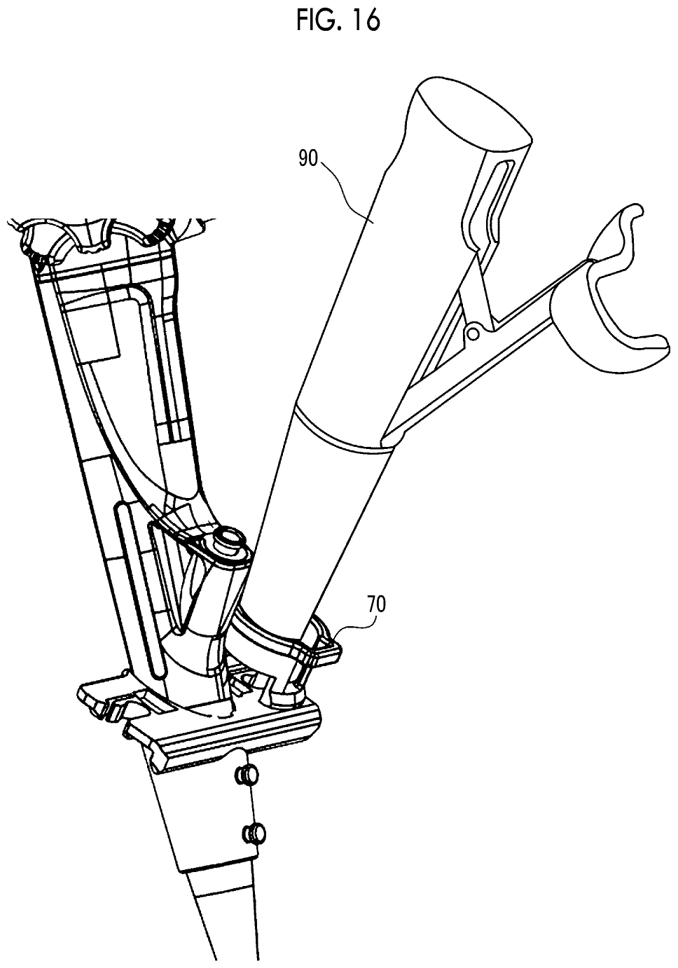

The adapter device 60 further has an attachment 70 that can hold a treatment tool 90 connected to the expansion unit 1A engaged with the second engagement portion 62. FIG. 15 is a perspective view showing a configuration example of the attachment 70. The attachment 70 has, for example, a cylindrical shape, and has two engaged portions 71 at one end portion thereof. A third engagement portion 63 that is engageable with the engaged portion 71 of the attachment 70 is provided around one of the two second engagement portions 62 in the flange portion 64 of the adapter device 60. The third engagement portion 63 has a structure that can be engaged with the engaged portion 71 of the attachment 70, for example, through a snap fit. The attachment 70 is engageable with the third engagement portion 63 around the second engagement portion 62 in a state in which the cylindrical member 40 is engaged with the second engagement portion 62. In a state in which the cylindrical member 40 is engaged with the second engagement portion 62 and the attachment 70 is engaged with the third engagement portion 63 around the second engagement portion 62, the third opening 43 of the cylindrical member 40 is disposed inside the attachment 70. In this state, a distal end part of a grip portion of the large-sized treatment tool 90 having a large grip portion is inserted into the attachment 70, and the third opening 43 is connected to the distal end part. As a result, it is possible to insert forceps or the like built in the grip portion of the treatment tool 90 into the first tubular member 30 and the pipe line 22. By using the attachment 70, the distal end part of the grip portion of the treatment tool 90 can be held by a cylindrical portion of the attachment 70, so that the large-sized treatment tool 90 can be stably disposed. As the large-sized treatment tool 90, for example, grip forceps with a bending function can be considered. FIG. 16 is a perspective view showing a state in which grip forceps with a bending function are additionally used in the endoscope 1. The grip forceps 90 with a bending function have a size close to that of the operation part 11, but even with such a large-sized treatment tool, the treatment tool can be stably held by providing the attachment 70.

As shown in FIG. 3, the third engagement portion 63 is provided at a position on a side opposite to the angle knob 12 in a state in which the adapter device 60 is mounted on the operation part 11. In the endoscope 1, since the operation part 11 is gripped by an operator's left hand, the third engagement portion 63 is provided on a side of an operator's right hand, so that the large-sized treatment tool 90 can be easily operated. The attachment 70 may be integrated with the flange portion 64.

The expansion unit 1A described above can be used in various ways. For example, in FIG. 3, a case is assumed in which the endoscope 1 is used by connecting the first connecting portion 51 to the mouthpiece 111A and opening the opening 51B via the lid part 51D. In this case, the treatment tool can be introduced into each of the treatment tool inlet port 112 and the third opening 43. As described above, the expansion unit 1A can function as an external treatment tool channel of the endoscope 1. Therefore, by using the expansion unit 1A, complex treatment which cannot be performed by a single treatment tool can be performed.

In addition, in FIG. 3, a case is assumed in which the first connecting portion 51 is connected to the mouthpiece 111A to close the opening 51B via the lid part 51D, and a forceps valve 54 is connected to the third opening 43 to close the third opening 43. The forceps valve 54 has the same configuration as that in which the sleeve part 51A and the lid part 51D in the first connecting portion 51 are integrated, and can be connected to the third opening 43 to close the third opening 43. In this case, the pipe line 22 of the expansion unit 1A and the suction channel inside the operation part 11 communicate with each other in a sealed state. In this state, in a case where the suction button 11A is operated, the fluid sucked into the pipe line 22 passes through the first tubular member 30, the cylindrical member 40, and the connecting member 50 to reach the suction channel inside the operation part 11, and can be collected by the above-described suction device. As described above, the expansion unit 1A can function as an external suction channel of the endoscope 1.

The expansion unit 1A can also be used by connecting the first connecting portion 51 to the third opening 43, in FIG. 3. In this case, it is impossible to suck the fluid via the expansion unit 1A, but it is possible to cause the expansion unit 1A to function as an external treatment tool channel.

It is preferable that the first opening 41, the second opening 42, and the third opening 43 communicate with each other in the cylindrical member 40, but the present invention is not limited thereto. For example, the second opening 42 and the third opening 43 do not need to be connected to each other. For example, a configuration may be adopted in which a lid part through which the treatment tool can be inserted but the fluid cannot pass is provided at the end part 44B of the cylindrical member 40, the first opening 41 and the third opening 43 can communicate with each other, and the third opening 43 and the second opening 42 are configured such that the treatment tool cannot be inserted therethrough.

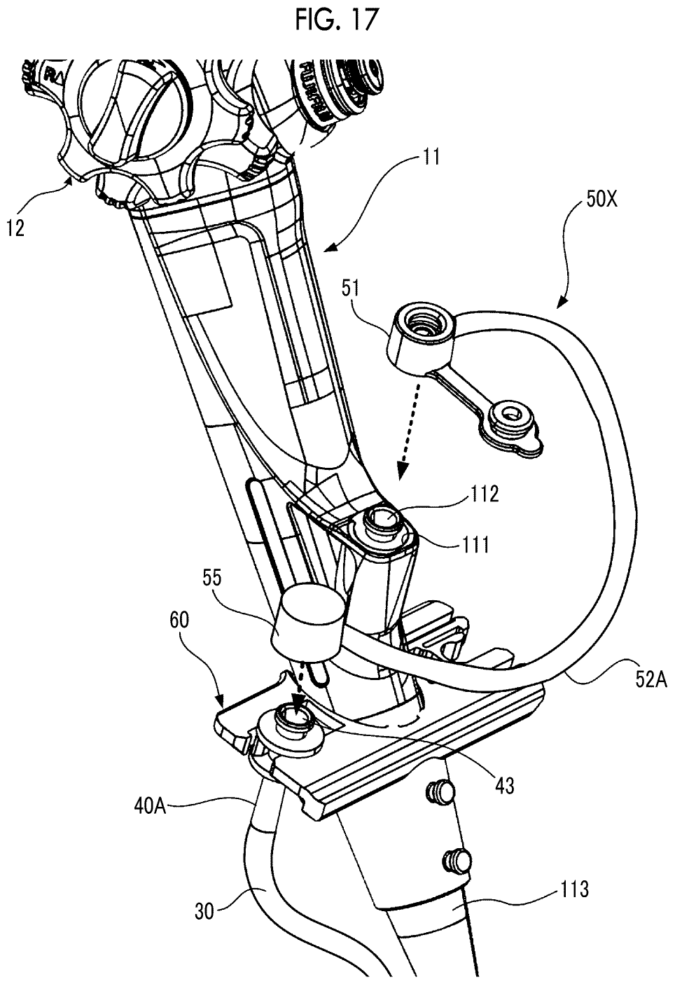

FIG. 17 is a diagram showing a modification example of the expansion unit 1A. The expansion unit 1A shown in FIG. 17 has a configuration in which the connecting member 50 is changed to a connecting member 50X and the cylindrical member 40 is changed to a cylindrical member 40A.

The connecting member 50X has a configuration in which the second tubular member 52 is changed to a second tubular member 52A and a second connecting portion 55 is added. The second tubular member 52A has a configuration in which the second connecting portion 55 is connected to one end thereof and the other end thereof is connected to the first connecting portion 51. The second connecting portion 55 has the same configuration as that in which the sleeve part 51A and the lid part 51D in the first connecting portion 51 are integrated, and can be connected to the third opening 43 to close the third opening 43. The cylindrical member 40A has a configuration in which the branch portion 45 shown in FIG. 4 is removed and only the main portion 44 is provided.

In the expansion unit 1A shown in FIG. 17, the second connecting portion 55 is connected to the third opening 43 of the cylindrical member 40A, the first connecting portion 51 is connected to the treatment tool inlet port 112, and in a state in which the opening 51B of the first connecting portion 51 is closed by the lid part 51D, the expansion unit 1A can function as an external suction channel. In addition, by removing the second connecting portion 55 from the third opening 43 of the cylindrical member 40A, a portion excluding the connecting member 50X in the expansion unit 1A can function as an external treatment tool channel. In the modification example shown in FIG. 17, the third opening 43 of the cylindrical member 40 constitutes a second opening, and the second tubular member 52A constitutes a third tubular member.

It is preferable that the expansion unit 1A described so far further has an elongated coating member that can collectively coat the distal end member 20 held by the distal end part 10C of the endoscope 1, the first tubular member 30, and the insertion part 10 of the endoscope 1. For example, in a state in which the distal end member 20 is engaged with the distal end part 10C, an elongated cylindrical coating member covers the endoscope 1 from the distal end side of the endoscope 1, and one end of the coating member is stretched to a position of the end part 113 of the operation part 11, so that the insertion part 10, the first tubular member 30, and the distal end member 20 are collectively coated. In this state, the first tubular member 30 and the insertion part 10 are fixed by winding a tape around an outside of the coating member at a plurality of positions in the axial direction of the endoscope 1, so that the first tubular member 30 can follow the movement of the insertion part 10.

As described above, at least the following matters are described in the present specification. Hereinafter, components corresponding to the above-described embodiment are shown in parentheses, but the present invention is not limited thereto.

(1)

An expansion unit (expansion unit 1A) of an endoscope (endoscope 1), comprising:
a distal end member (distal end member 20) that is configured to be able to be held at a distal end part (distal end part 10C) of the endoscope (endoscope 1);
a first tubular member (first tubular member 30) that extends from the distal end member;
a cylindrical member (cylindrical member 40) that has a first opening (first opening 41), that is connected to a base end part (base end part 32) of the first tubular member opposite to a side of the distal end member, and a second opening (second opening 42); and
a connecting member (connecting member 50) that connects the second opening and a treatment tool inlet port (treatment tool inlet port 112) of the endoscope.
   (2)

The expansion unit according to (1),
in which the connecting member has a first connecting portion (first connecting portion 51) connected to the treatment tool inlet port and a second tubular member (second tubular member 52) having one end connected to the second opening and the other end connected to the first connecting portion.
   (3)

The expansion unit according to (2),
in which the first connecting portion has a sleeve part (sleeve part 51A) connected to the treatment tool inlet port, and the sleeve part is configured to be able to generate a negative pressure inside in a case where a suction device connected to the endoscope is operated.
   (4)

The expansion unit according to any one of (1) to (3),
in which the cylindrical member has a third opening (third opening 43) leading to at least the first opening among the first opening and the second opening.
   (5)

The expansion unit according to (4), in which the cylindrical member has a first hollow portion (passage 44A) extending from the first opening to the third opening and a second hollow portion (branch path 45A) extending from the first hollow portion to the second opening.

(6)

The expansion unit according to (5),
in which the connecting member is configured to be able to connect the third opening and the second opening.
   (7)

The expansion unit according to (1),
in which the connecting member has a first connecting portion (first connecting portion 51) connected to the treatment tool inlet port, a second connecting portion (second connecting portion 55) connected to the second opening (third opening 43), and a third tubular member (second tubular member 52A) having one end connected to the second connecting portion and the other end connected to the first connecting portion.
   (8)

The expansion unit according to any one of (1) to (7),
in which the distal end member has an engagement portion (engagement portion 21) that engages with the distal end part of the endoscope, and a pipe line (pipe line 22) that is disposed radially adjacent to the distal end part and that communicates with the first tubular member in a state in which the engagement portion is engaged with the distal end part.
   (9)

The expansion unit according to (8),
in which the pipe line includes a pipe line extending part (pipe line extending part 22A) that extends further to a base end side of the endoscope than the engagement portion.
   (10)

The expansion unit according to (9),
in which the first tubular member is connected to the pipe line extending part.
   (11)

The expansion unit according to (9) or (10),
in which the distal end member has a distal end member extending part (distal end member extending part 23) that extends from the engagement portion to the base end side of the endoscope.
   (12)

The expansion unit according to (11),
in which a length (length L2) in an extending direction of the distal end member extending part is equal to or longer than a length (length L1) in an extending direction of the pipe line extending part.
   (13)

The expansion unit according to (11) or (12),
in which the distal end member extending part is provided adjacent to the endoscope that is engaged with the engagement portion and to the pipe line extending part.
   (14)

The expansion unit according to (13),
in which the distal end member extending part is provided across a line segment (line segment L3) connecting a center of the endoscope and a center of the pipe line extending part in a case of being viewed in an axial direction of the endoscope that is engaged with the engagement portion.
   (15)

The expansion unit according to (14),
in which the distal end member extending part includes a curved surface (second curved surface 23A) along an outer peripheral surface of the distal end part of the endoscope that is engaged with the engagement portion.
   (16)

The expansion unit according to (8),
in which the distal end member has a distal end member extending part (distal end member extending part 23) that extends from the engagement portion to a base end side of the endoscope.
   (17)

The expansion unit according to any one of (8) to (16),
in which the engagement portion and the pipe line are integrally formed.
   (18)

The expansion unit according to any one of (8) to (17),
in which the distal end member has a protruding portion (protruding portion 24) that protrudes from the engagement portion toward an imaging direction (direction X1) of the endoscope that is engaged with the engagement portion.
   (19)

The expansion unit according to any one of (1) to (18),
in which the cylindrical member is configured to be able to engage with a part of an adapter device (adapter device 60) mounted on an operation part (operation part 11) of the endoscope.
   (20)

The expansion unit according to any one of (1) to (19), further comprising:
a coating member that is configured to be able to coat an insertion part (insertion part 10) of the endoscope, the distal end member held by the distal end part of the endoscope, and the first tubular member.
   (21)

An adapter device (adapter device 60) of an endoscope, comprising:
a first engagement portion (first engagement portion 61) that attachably and detachably engages with an operation part (operation part 11) of the endoscope (endoscope 1); and
a second engagement portion (second engagement portion 62) that attachably and detachably engages with an expansion unit (expansion unit 1A) that adds a channel to the endoscope.
   (22)

The adapter device according to (21),
in which the first engagement portion is formed of a cylindrical body,
the adapter device further comprises a flange portion (flange portion 64) that protrudes from the first engagement portion, and
the second engagement portion is provided on the flange portion.
   (23)

The adapter device according to (22),
in which the flange portion is inclined with respect to an axis (axis AX) of the first engagement portion.
   (24)

The adapter device according to (22) or (23),
in which the flange portion has a shape along an opening surface (opening surface 112A) of a treatment tool inlet port (treatment tool inlet port 112) of the endoscope in a state in which the first engagement portion is engaged with the operation part of the endoscope.
   (25)

The adapter device according to any one of (22) to (24),
in which the flange portion protrudes from an edge (opening 61E) of the first engagement portion on a base end side of the endoscope in a state in which the first engagement portion is engaged with the operation part of the endoscope.
   (26)

The adapter device according to any one of (22) to (25),
in which the flange portion is disposed on a distal end side of the endoscope with respect to a treatment tool inlet port (treatment tool inlet port 112) of the operation part in a state in which the first engagement portion is engaged with the operation part of the endoscope.
   (27)

The adapter device according to any one of (22) to (26),
in which a plurality of the second engagement portions are provided, and the second engagement portions are disposed to face each other with the first engagement portion interposed therebetween.
   (28)

The adapter device according to any one of (22) to (27), further comprising:
an attachment (attachment 70) that is configured to be able to hold a treatment tool (treatment tool 90) connected to the expansion unit that is engaged with the second engagement portion,
in which a third engagement portion (third engagement portion 63) that is configured to be able to engage with the attachment is provided on the flange portion.
   (29)

The adapter device according to (28),
in which a plurality of the second engagement portions are provided,
the second engagement portions are disposed to face each other with the first engagement portion interposed therebetween, and
the third engagement portion is provided around any one of the plurality of second engagement portions.
   (30)

The adapter device according to any one of (22) to (29),
in which a locking portion (protrusion 65) that is configured to be able to lock a band-shaped body (fixing tool 80) is provided on an outer peripheral surface of the first engagement portion.
   (31)

The adapter device according to any one of (22) to (30),
in which a hole portion (flange hole portion 64H) into which a band-shaped body (fixing tool 80) is capable of being inserted is provided between the first engagement portion and the second engagement portion in the flange portion.

### EXPLANATION OF REFERENCES

1: endoscope
1A: expansion unit
2: body part
L3: line segment
5: processor device
4: light source device
7: display device
10: insertion part
10A: soft part
10B: bendable part
10C, 31: distal end part
11: operation part
12: angle knob
13: universal cord
13A: connector portion
20: distal end member
21: engagement portion
21A: space
22: pipe line
22A: pipe line extending part
23: distal end member extending part
23A: second curved surface
23B: first curved surface
23C, 23D: plane
24: protruding portion
30: first tubular member
32: base end part
40, 40A: cylindrical member
41: first opening
42: second opening
43: third opening
44: main portion
44A: passage
44B, 44C, 113: end part
45: branch portion
45A: branch path
46: first flange portion
47: second flange portion
48, 71: engaged portion
50, 50X: connecting member
51: first connecting portion
51A: sleeve part
51B, 51C, 61A, 61E, 111: opening
51D: lid part
52, 52A: second tubular member
55: second connecting portion
60: adapter device
61: first engagement portion
62: second engagement portion
63: third engagement portion
64: flange portion
64H: flange hole portion
64S: surface
65: protrusion
70: attachment
80: fixing tool
90: treatment tool
100: endoscope device
111A: mouthpiece
112: treatment tool inlet port
112A: opening surface
210: bulging part
210A: hole portion

What is claimed is:

1. An expansion unit of an endoscope, comprising:
a distal end member that is configured to be able to be held at a distal end part of the endoscope;
a first tubular member that extends from the distal end member;
a cylindrical member that has a first opening, that is connected to a base end part of the first tubular member opposite to a side of the distal end member, and a second opening; and
a connecting member that connects the second opening and a treatment tool inlet port of the endoscope.

2. The expansion unit according to claim 1,
wherein the connecting member has a first connecting portion connected to the treatment tool inlet port and a second tubular member having one end connected to the second opening and other end connected to the first connecting portion.

3. The expansion unit according to claim 2,
wherein the first connecting portion has a sleeve part connected to the treatment tool inlet port, and the sleeve part is configured to be able to generate a negative pressure in the sleeve part in a case where a suction device connected to the endoscope is operated.

4. The expansion unit according to claim 1,
wherein the cylindrical member has a third opening leading to at least the first opening among the first opening and the second opening.

5. The expansion unit according to claim 4,
wherein the cylindrical member has a first hollow portion extending from the first opening to the third opening and a second hollow portion extending from the first hollow portion to the second opening.

6. The expansion unit according to claim 5,
wherein the connecting member is configured to be able to connect the third opening and the second opening.

7. The expansion unit according to claim 1,
wherein the connecting member has a first connecting portion connected to the treatment tool inlet port, a second connecting portion connected to the second opening, and a third tubular member having one end connected to the second connecting portion and other end connected to the first connecting portion.

8. The expansion unit according to claim 1,
wherein the distal end member has an engagement portion that engages with the distal end part of the endoscope, and a pipe line that is disposed radially adjacent to the distal end part and that communicates with the first tubular member in a state in which the engagement portion is engaged with the distal end part.

9. The expansion unit according to claim 8,
wherein the pipe line includes a pipe line extending part that extends further to a base end side of the endoscope than the engagement portion.

10. The expansion unit according to claim 9,
wherein the first tubular member is connected to the pipe line extending part.

11. The expansion unit according to claim 10,
wherein the distal end member has a distal end member extending part that extends from the engagement portion to the base end side of the endoscope.

12. The expansion unit according to claim 11,
wherein a length in an extending direction of the distal end member extending part is equal to or longer than a length in an extending direction of the pipe line extending part.

13. The expansion unit according to claim 11,
wherein the distal end member extending part is provided adjacent to the endoscope that is engaged with the engagement portion and to the pipe line extending part.

14. The expansion unit according to claim 13,
wherein the distal end member extending part is provided across a line segment connecting a center of the endoscope and a center of the pipe line extending part in a case of being viewed in an axial direction of the endoscope that is engaged with the engagement portion.

15. The expansion unit according to claim 14,
wherein the distal end member extending part includes a curved surface along an outer peripheral surface of the distal end part of the endoscope that is engaged with the engagement portion.

16. The expansion unit according to claim 8,
wherein the distal end member has a distal end member extending part that extends from the engagement portion to a base end side of the endoscope.

17. The expansion unit according to claim 8,
wherein the engagement portion and the pipe line are integrally formed.

18. The expansion unit according to claim 8,
wherein the distal end member has a protruding portion that protrudes from the engagement portion toward an imaging direction of the endoscope that is engaged with the engagement portion.

19. The expansion unit according to claim 1,
wherein the cylindrical member is configured to be able to engage with a part of an adapter device mounted on an operation part of the endoscope.

20. The expansion unit according to claim 1, further comprising:
a coating member that is configured to be able to coat an insertion part of the endoscope, the distal end member held by the distal end part of the endoscope, and the first tubular member.

21. An adapter device of an endoscope, comprising:
a first engagement portion that attachably and detachably engages with an operation part of the endoscope; and
a second engagement portion that attachably and detachably engages with an expansion unit that adds a channel to the endoscope.

22. The adapter device according to claim 21,
wherein the first engagement portion is formed of a cylindrical body,
the adapter device further comprises a flange portion that protrudes from the first engagement portion, and
the second engagement portion is provided on the flange portion.

23. The adapter device according to claim 22,
wherein the flange portion is inclined with respect to an axis of the first engagement portion.

24. The adapter device according to claim 23,
wherein the flange portion has a shape along an opening surface of a treatment tool inlet port of the endoscope in a state in which the first engagement portion is engaged with the operation part of the endoscope.

25. The adapter device according to claim 22,
wherein the flange portion protrudes from an edge of the first engagement portion on a base end side of the endoscope in a state in which the first engagement portion is engaged with the operation part of the endoscope.

26. The adapter device according to claim 22,
wherein the flange portion is disposed on a distal end side of the endoscope with respect to a treatment tool inlet port of the operation part in a state in which the first engagement portion is engaged with the operation part of the endoscope.

27. The adapter device according to claim 22,
wherein a plurality of the second engagement portions are provided, and
the second engagement portions are disposed to face each other with the first engagement portion interposed therebetween.

28. The adapter device according to claim 22, further comprising:
an attachment that is configured to be able to hold a treatment tool connected to the expansion unit that is engaged with the second engagement portion,
wherein a third engagement portion that is configured to be able to engage with the attachment is provided on the flange portion.

29. The adapter device according to claim 28,
wherein a plurality of the second engagement portions are provided,
the second engagement portions are disposed to face each other with the first engagement portion interposed therebetween, and
the third engagement portion is provided around one of the plurality of second engagement portions.

30. The adapter device according to claim 22,
wherein a locking portion that is configured to be able to lock a band-shaped body is provided on an outer peripheral surface of the first engagement portion.

31. The adapter device according to claim 22,
wherein a hole portion into which a band-shaped body is capable of being inserted is provided between the first engagement portion and the second engagement portion in the flange portion.
